(19) United States
(12) Patent Application Publication
(10) Pub. No.: US 2011/0201961 A1
(43) Pub. Date: Aug. 18, 2011

(54) MORPHOLOGICAL CLUSTERING AND ANALYSIS OF INTRACRANIAL PRESSURE PULSES (MOCAIP)

(75) Inventors: Xiao Hu, Redondo Beach, CA (US); Marvin Bergsneider, Los Angeles, CA (US)

(73) Assignee: THE REGENTS OF THE UNIVERSITY OF CALIFORNIA, Oakland, CA (US)

(21) Appl. No.: 12/985,603

(22) Filed: Jan. 6, 2011

Related U.S. Application Data
(63) Continuation of application No. PCT/US2009/053602, filed on Aug. 12, 2009.
(60) Provisional application No. 61/088,114, filed on Aug. 12, 2008.

(51) Int. Cl. A61B 5/00

(52) U.S. Cl. 600/561

(57) ABSTRACT

A system and method for recognizing the locations of the three ICP sub-peaks present in Intracranial Pressure (ICP) pulses and then calculating pulse metrics automatically and continuously. These metrics allow a comprehensive quantitative characterization of ICP pulse morphology including pulse amplitude, time intervals among sub-peaks, curvature, slope, and decay time constants over a course of time. One embodiment of the system provides real time monitoring and forecasting of intracranial and cerebrovascular pathophysiological changes with beat-by-beat pulse detection, pulse clustering, non-artifactual pulse recognition, peak detection and optimal peak designation processes.

### CROSS-REFERENCE TO RELATED APPLICATIONS

This application a 35 U.S.C. §111(a) continuation of, and claims priority to, PCT international application number PCT/US2009/053602 filed on Aug. 12, 2009, incorporated herein by reference in its entirety, which claims priority to U.S. provisional application Ser. No. 61/088,114 filed on Aug. 12, 2008, which is incorporated herein by reference in its entirety.

The above-referenced PCT international application was published on Feb. 18, 2010 as PCT International Publication No. WO 2010/019705 (republished on Jun. 17, 2010), and is incorporated herein by reference in its entirety.

### STATEMENT REGARDING FEDERALLY SPONSORED RESEARCH OR DEVELOPMENT

This invention was made with Government support under Grant Nos. NS054881, NS055045, and NS055998 awarded by the National Institutes of Health. The Government has certain rights in this invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to intracranial pressure diagnostic and monitoring detectors, and more particularly to a system and method for continuous Intracranial Pressure Pulse (ICP) signal analysis and tracking of pulse metrics for real time diagnosis and prospective treatments.

2. Description of Related Art

The treatment of many neurological disorders and brain injuries relies on the continuous measurement of different physiological signals like Electrocardiogram (ECG), Intracranial Pressure (ICP), Saturation of Peripheral Oxygen (Spo2), and Arterial Blood Pressure (ABP) by physicians. Dynamic changes in the intracranial pressure (ICP) reflect the ability of the body to compensate for changes in volume within the skull and pathophysiological changes in the cerebral vasculature.

Raised intracranial pressure and low cerebral blood flow are common indicators associated with ischaemia and correlated with high morbidity and mortality after a brain injury. Since the brain is encased in a skull that does not expand and the brain parenchyma is essentially incompressible, the volume of fluids in the cranium is essentially constant and at an equilibrium. Thus, the outflow of venous blood leaving the cranial cavity is approximately the volume of arterial blood entering the cranial cavity. Compensatory mechanisms that reduce the volume of intracranial blood or cerebrospinal fluid (CSF) are also present to maintain ICP homeostasis.

ICP monitoring is therefore an essential element of current treatment protocols. The conventional method for assessing ICP is with the surgical insertion of a catheter into one of the lateral ventricles of the brain that is then connected to an external pressure transducer. The output of the transducer produces a characteristic ICP pulse waveform. It has been recognized that an ICP pulse is typically triphasic with three subpeaks that originate mostly from cerebral arterial pulsations with some contributions of venous origin. Generally, ICP pulse wave forms have three characteristic peaks that are referred to a P1 (percussion wave), P2 (tidal wave) and P3 (dicrotic wave). The P1 peak normally sharp, with constant amplitude and has its origin in the choroid plexis. The second (P2) peak is rebound after the arterial percussion and ends in the dicrotic notch. The third peak is venous in origin.

Changes in the amplitude and configuration of the ICP pulse can reflect changes in the physiological conditions within the skull such as the cerebral autoregulation or intracranial adaptive capacity or elastance. The amplitude of the ICP pulse waveform is the response of the intracranial pressure to a volume increase and craniospinal elastance.

Despite the importance of ICP monitoring, signal processing capabilities in existing commercial ICP monitoring devices remain poor providing clinicians with a limited amount of information that is confined to the mean ICP. As a consequence, clinical decisions related to treating ICP-related abnormalities are typically made solely based on mean ICP although raw continuous waveform data are usually available. The utilization of only mean ICP, however, ignores the potentially rich information embedded in dynamics of ICP that may be related to cerebral volume compensatory mechanism and cerebral vascular pathophysiology.

Accordingly, there is a need for a system and method for ICP monitoring that not only evaluates mean ICP, but can continuously evaluate the dynamic morphological features of the ICP pulse wave forms that is at the same time accurate, reliable and computationally practical. The present methods satisfy these needs, as well as others, and are generally an improvement over the art.

### BRIEF SUMMARY OF THE INVENTION

The present invention provides a system and method for automatic and continuous monitoring of intracranial pulse (ICP) characteristics and to track changes in pulse morphology over time. Continuous monitoring and analysis of ICP pulse wave form characteristics permit the accurate diagnosis, treatment and forecasting of intracranial and cerebrovascular pathophysiological changes in a patient. By way of example, and not of limitation, a system and method is provided for the continuous acquisition and analysis of refined ICP Pulses with identification of the locations of the three ICP sub-peaks and then calculating as many as 24 metrics that can be tracked and evaluated as an illustration. These metrics allow a comprehensive quantitative characterization of ICP pulse morphology including pulse amplitude, time intervals among sub-peaks, curvature, slope, and decay time constants.

Generally, the method accomplishes this detailed analysis of ICP pulse by sampling a discrete period of a digital ICP recording, and then in order: 1) performing individual ICP pulse detection, 2) segregating ICP pulses using cluster analysis, and then 3) rejecting individual illegitimate (incomplete, bizarre, etc) waveforms. The latter step is preferably facilitated by comparison to a library of legitimate ICP pulses derived from a large patient database, in one embodiment. Representative ICP waveforms from each cluster group are preferably derived by an averaging process, which greatly improves the signal-to-noise ratio. The representative waveform from this dominant cluster is then used for sub-peak detection and designation. The peak designations are achieved by two different methods. The first method uses the Gaussian prior of each peak's distribution to help designation of each sub peaks in an optimal way. The second method poses the problem of peak designation as a regression problem and solves it using a nonlinear regression approached such as kernel spectral regression.

According to one embodiment of the invention, ICP pulse data is acquired, processed and displayed for physician evaluation and treatment decisions from sensors and computers as follows:

Pulse Detection. ICP Pulses are preferably acquired using both ECG and ICP signals as input. As a result, the start of individual ICP pulses is defined at the corresponding QRS peak of ECG. The raw ICP Pulse signals that are acquired are preferably recorded and stored in the apparatus.

Pulse Clustering. Clinical ICP recordings are often contaminated by noise and artifacts including instrument noise, transient perturbations, sensor detachment, and quantization noise by the digitization process. These noises and artifacts result in poor quality of individual ICP pulses that hamper a detailed analysis of their morphological features. It is therefore preferred that the analysis of ICP pulse morphology take place by using a representative cleaner pulse to be extracted from a sequence of consecutive raw ICP pulses rather than each individual pulse. The apparatus algorithm preferably uses a clustering method to extract this representative ICP pulse. A sequence of raw ICP pulses are first clustered into distinct groups based on their morphological distance. The largest cluster is then identified. An averaging process is conducted to obtain an averaged pulse for this largest cluster. This average pulse of the largest cluster is called a dominant ICP pulse. Subsequent analysis of ICP morphology will be only conducted for this dominant pulse.

Legitimate Pulse Recognition. A dominant pulse is immune to noises of a transient nature. However, the pulse could still be artifactual because the complete segment it represents could be noise, e.g., sensor detachment can cause several minutes or even hours of an ICP recording to be invalid. To identify legitimate ICP pulses automatically, legitimate pulses are verified. In one preferred embodiment, a filtering that is based on two verifications that both uses a second hierarchical clustering applied on the dominant pulses previously found by the hierarchical pulse clustering. The first verification exploits a reference library containing validated ICP pulses that have been manually extracted from data of multiple patients. A pulse is judged to be legitimate if it belongs to a cluster whose average pulse correlates with any of the reference ICP pulses.

The second test measures the coherence of a cluster using the average of the correlation coefficients between each member to the average pulse of the cluster. The dominant pulses of the cluster that fail both checks are considered to be illegitimate and are excluded from further analysis.

Detection of ICP Sub-peaks. Instead of using the strict condition xᵢ₋₁<xᵢ<xᵢ₊₁ to define position i as a peak, the algorithm performs a comprehensive search for all landmark points on an ICP pulse as candidates for designating the three ICP pulse sub-peaks. The first step to finding the landmarks is to calculate the second derivative of an ICP pulse. Based on the sign of the second derivative, an ICP pulse can be segmented into concave and convex regions. The intersection of a concave to a concave region on the ascending portion of the pulse is treated as a landmark. On the descending portion of the pulse, the intersection of a convex to a concave region is also treated as a landmark in this embodiment.

Assignment of Detected Peaks. The objective of the last step of the MOCAIP algorithm is to obtain the best designation of the three well-recognized ICP sub-peaks, denoted as P₁, P₂, and P₃ respectively, from an array of detected candidate peaks plus an empty designation. Where a₁, a₂, . . . , a_{N} represents an array of N detected peak candidates and a₀ represents an empty designation such that if a₀ is assigned to one of P₁, P₂, and P₃, it means that no corresponding sub-peak is present.

Analysis of Peaks And Metrics. The refined ICP pulse form can be analyzed and patterns can be observed and evaluated over time. The peaks and calculated metrics can be indicators of existing conditions or may forecast other conditions in the patient that can be monitored, recorded and displayed over the course of treatment. Patterns of metrics exhibited by a patient can be compared with patterns of metrics seen with documented conditions in many other patients to form a library of patterns for comparison. Patient conditions can be determined and treatment decisions can be made and implemented in a very short time frame.

According to another embodiment of the invention, a method for extracting morphological features from intracranial pressure pulses is provided that has the process steps of acquiring intracranial pressure pulse data of a patient from at least one sensor; refining the acquired pulse data with a computer and programming to produce refined pulse data; and then determining peaks and metrics from the refined pulse data.

A further embodiment of the invention provides a system and method for extracting morphological features from intracranial pressure pulses by obtaining intracranial pressure pulse data of a patient from a sensor; and processing the obtained pressure pulse data with a computer with the steps of clustering the pulse data to produce a plurality of dominant pulses; validating the dominant pulses to eliminate false dominant pulses; detecting at least one subcomponent peak within the dominant pulses; designating final peaks and metrics of said dominant pulses; and then analyzing the designated peaks and metrics.

Yet another embodiment of the invention provides a system and method for extracting morphological features from intracranial pressure pulses for patient treatment that acquires intracranial pressure pulse data from a patient from a plurality of intracranial pressure (ICP) pulse and electrocardiogram (ECG) sensors and processes the intracranial pressure pulse data with a computer by clustering the pulse data to produce a plurality of dominant pulses; validating the dominant pulses to eliminate false dominant pulses; detecting at least one subcomponent peak within said dominant pulses; designating final peaks and metrics of said dominant pulses; analyzing the designated peaks and metrics; and then comparing the analyzed and designated peaks and metrics of the patient with analyzed and designated intracranial pressure pulse peaks and metrics of one or more previous patients and predicting possible physiological conditions and events of the patient from said comparison of said peaks and metrics.

According to one aspect of the invention, a system is provided that records intracranial pressure pulse peaks and metrics obtained from intracranial pulse data of a patient over a course of time; correlates the patient symptoms and conditions with pulse peaks and metrics over the course of time; and forms a profile of correlated data for comparison with current patient data.

According to another aspect of the invention, a library of patent profiles and data is provided that permits the identification of patterns of correlated symptoms, pulse peaks and metrics over time.

According to another aspect of the invention, a method is provided for the continuous and automatic display of ICP pulse data, peaks and metrics for real time status evaluation and diagnosis.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a flow diagram of a method for ICP Pulse refinement and morphological feature extraction according to one embodiment of the invention.

FIG. 2A depicts an illustrative refined ICP pulse with 24 metrics that can be extracted and monitored according to the invention.

FIG. 2B depicts the metrics that can be calculated and shown in the refined ICP pulse in FIG. 2A.

### DETAILED DESCRIPTION OF THE INVENTION

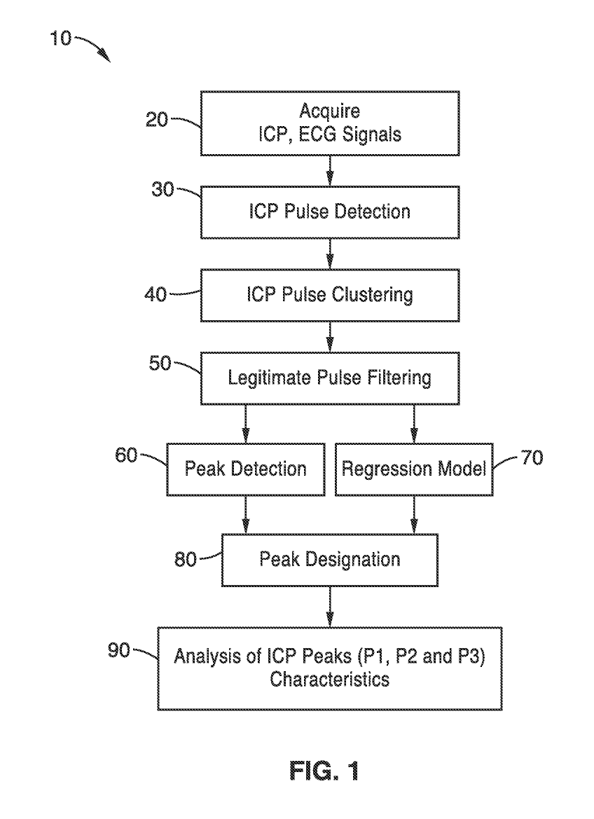

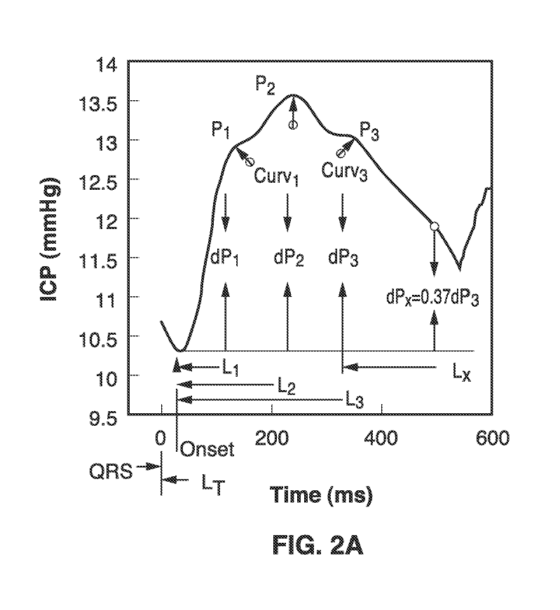

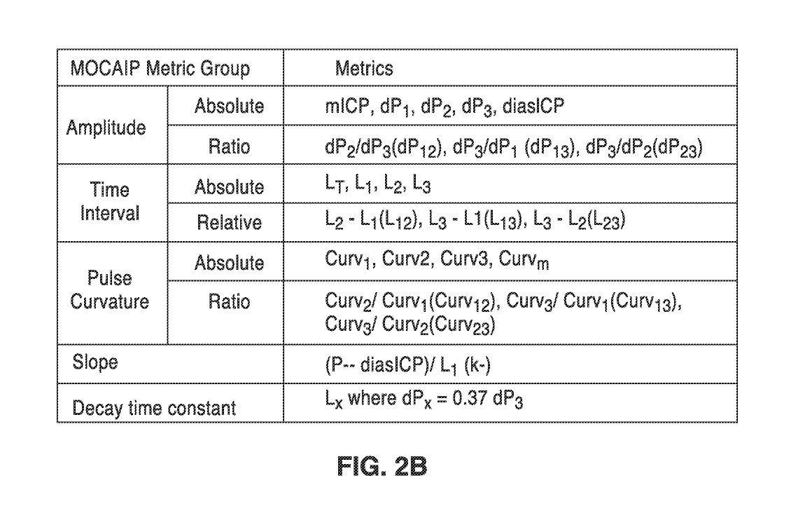

Referring more specifically to the drawings, for illustrative purposes the present invention is embodied in the methods generally shown in FIG. 1 through FIG. 2A and the associated devices used to perform the methods. It will be appreciated that the apparatus may vary as to configuration and as to details of the parts, and that the method may vary as to the specific steps and sequence, without departing from the basic concepts as disclosed herein.

The present invention relates to an improved intracranial pressure monitoring apparatus and system for the continuous morphological analysis of ICP pulses. The preferred method uses the Morphological Cluster and Analysis of Intracranial Pressure (MOCAIP) algorithm to extract pulse morphological metrics for evaluation by the physician.

Turning now to the flow diagram show in FIG. 1, one embodiment 10 of the invention is schematically shown. In FIG. 1, intracranial pulse signals are acquired at block 20 from a sensor using conventional methods of installation. Signals produced from the sensors are preferably recorded and analyzed or may be stored in random access memory of a computer and analyzed in real time without recording in the alternative. In the preferred embodiment, the continuous input signals optionally include ECG signals to produce a stream of pulse data.

The acquired pulse signal data at Block 20 is processed with a number of process steps to eliminate noise and to refine the peaks for analysis at Block 90. In the embodiment shown in FIG. 1, a system with five major components is provided including a beat-by-beat pulse detection component 30, a pulse clustering component 40, a non-artifactual pulse recognition component 50, a peak detection component 60, and an optimal peak designation component 80. In addition, the algorithm makes use of a library of reference ICP pulses that contains a collection of pulses and locations of their designated three peaks. The beat-by-beat detection of the ICP pulse at Block 30 is preferably conducted using an algorithm developed in X. Hu, P. Xu, D. J. Lee, P. Vespa, K. Baldwin, and M. Bergsneider, “An algorithm for extracting intracranial pressure latency relative to electrocardiogram r wave,” Physiol. Meas., vol. 29, no. 4, pp. 459-471, 2008, incorporated by reference.

Pulse clustering may be used in two stages of processing. Clustering is initially applied to consecutive subsequences of the raw ICP pulses obtained from the ICP pulse detection process to generate a dominant pulse for each pulse sequence at Block 40. This process results in a sequence of dominant ICP pulses that is further analyzed by the pulse recognition component 50. Pulse clustering may be applied again to the sequence of dominant pulses in this process. The recognized non-artifactual pulses may be further processed to detect all peak candidates in each of them. Finally, the peak designation process 80 is executed to optimally designate the three well-established ICP peaks in each non-artifactual dominant pulse using the detected peak candidates in the embodiment shown.

Referring now to Block 30, the ICP pulse is detected from the ICP signals from the sensors. This step segments the continuous ICP into a sequence of individual ICP pulses. Instead of solely using ICP for pulse detection, the mature technique of ECG QRS detection to first find each ECG beat is preferred to achieve reliable ICP pulse detection. Optionally, interval constraints for ICP peak locations can be incorporated to prevent false ICP pulse detections that would be caused by spurious ECG QRS detections. The interval constraints can also be adapted on a beat-by-beat basis.

ICP recordings collected from bedside monitors can often be contaminated by several types of noise and artifacts. For example ICP pulses can be contaminated by high-frequency noise that originated from measurement or amplifier devices. Transient artifacts from coughing or patient movement or ICP recordings with the sensor detached from the patient monitor for a period of time. These artifacts and noise are common for typical ICP recordings and can interfere with the analysis of ICP pulse morphology.

Instead of applying the ICP morphology analysis to each individual pulse separately, a representative cleaner pulse is preferably extracted from a sequence of consecutive ICP pulses at Block 40. Therefore, a continuous ICP recording can be segmented into consecutive pulse sequences and morphological characteristics of the pulses can be calculated based on the representative pulse of each sequence, in this embodiment.

In one embodiment at block 40, a sequence of raw ICP pulses is first clustered into distinct groups based on their morphological distance. The largest cluster is then identified. An averaging process is conducted to obtain an averaged pulse for this largest cluster. These averaged pulses of the largest cluster are called dominant ICP pulses. Subsequent analysis of ICP morphology will be only conducted for this dominant pulse. This dominant pulse is preferred for performing morphological analysis because the clustering procedure will effectively isolate transient disturbances from the normal ICP pulses. Therefore, the dominant ICP pulse would most likely represent the signal group. In addition, the averaging process effectively reduces influences from random noise and quantization noise on the morphological analysis of the ICP pulse by enhancing the signal-to-noise ratio.

In one embodiment, a hierarchical clustering approach is used to cluster ICP pulses at Block 40 because it does not require a prior specification of the number of clusters. After the clustering procedure, the largest cluster is retained to extract the dominant pulse.

It can be seen that a dominant pulse is immune to noises of a transient nature. However, dominant pulse clusters extracted from signal segments could still be artifactual because the complete segment it represents could be noise. For example, sensor detachment can cause several minutes or even hours of ICP recording to be invalid. In such cases, the dominant pulses should not be analyzed any further.

To identify legitimate dominant ICP pulses in an automated fashion, a reference library of validated ICP pulses is preferably used to aid the recognition of non-artifactual peaks at Block 50. This library of reference ICP pulses is preferably constructed with legitimate pulses of divergent shapes. The library preferably uses data sets from many different patients. In one embodiment, a self-identification component is incorporated so that a non-artifactual ICP pulse that does not match a template found in the library is not falsely rejected. For example, a self-authentication may be created by further clustering the dominant pulses found in the first pass of the clustering analysis since a cluster formed by an artifactual dominant pulses will be less coherent that a cluster formed by non-artifactual pulses.

The input at Block 50 is the sequence of dominant pulses identified for each consecutive sub-sequence of the signal segment being processed. This sequence may be further clustered. The average dominant pulse of each cluster is then subject to a matching test with each reference pulse found in the library with a correlation analysis. A dominant pulse is considered to be a non-artificial pulse if it belongs to a cluster that has an average pulse that correlates with any of the reference ICP pulses with a correlation coefficient greater than a selected value, for example, a correlation coefficient greater than r₁. To avoid the false rejection of a valid cluster because of the incompleteness of the reference library or inappropriate r₁, those clusters that fail the first test will be further checked by comparing its self coherence against r₂. Accordingly, the dominant pulses of the cluster that fails both checks will be excluded from further analysis in this embodiment.

Once a valid ICP pulse has been extracted and verified at Block 50 a set of peak candidates (or curve inflections) are detected at Block 60 of FIG. 1. Each candidate is potentially one of the three peaks. The extraction of these candidates relies on the segmentation of the ICP pulse form into concave and convex regions. This is preferably accomplished using the second derivative of the pulse.

Generally, peak locations may be found at Block 60 using the concave portions of the pulse curve according to four possible definitions in the embodiment shown. The first definition treats the intersection of a concave to a convex region as a peak if the first derivative of the concave portion is greater than zero otherwise the intersection of a convex region to a concave region is the peak. The second definition is based on the curvature of the signal such that the peak is the location with maximal absolute curvature within each concave region, the third and the fourth definitions both involve a straight line linking the two end points of a concave region. According to the third and the fourth definitions, a peak can be found at the position where the perpendicular distance or the vertical distance from the ICP to this line is maximal, respectively.

Typically, a peak corresponds to the intersection of a convex to a concave region on a rising edge of ICP pulse or to the intersection of a concave to a convex region on the descending edge of the pulse. This detection process at Block 60 produces a pool of N peak candidates (a₁, a₂, . . . , a_{N}).

At Block 80 of FIG. 1, the detected peaks are assigned. The objective of Block 80 is to obtain the best designation of the three well-recognized ICP peaks, denoted as P₁, P₂ and P₃, respectively, from an array of detected candidate peaks at Block 60. Given Pi(aj), i=1, 2, 3 to denote the probability density functions (PDF) of assigning aj to the i-th peak (each PDF is a Gaussian distribution estimated from peak locations previously detected on a set of reference ICP pulses). In order to deal with missing peaks, an empty designation a0 is added to the pool of candidates. In addition, to avoid false designation, MOCAIP uses a threshold q such that Pi(ak)=0, i [{1, 2, 3}, k [{1, 2, . . . , N} if the probability of assigning ak to pi is less than q.

In an alternative embodiment, the detection and assignment of peaks is accomplished with a regression model at Block 70 instead of using unimodal priors during peak designation to improve the accuracy of the peak designation process.

Referring now to Block 70 and Block 80, a regression model y=f(x) is able to predict the most likely position of the three peaks, y=(p1, p2, p3), given a segmented ICP pulse discretized as a vector x. Regression analysis is a statistical technique used for the numerical analysis between an input variable and an output variable. Different regression analysis methods may be used such as Multi-Linear Regression, Support vector machine (SVM) algorithm, spectral regression (SR) analysis, and extremely randomized decision trees.

During the peak assignment at Block 80, the method exploits Gaussian priors to infer the position of the three peaks from a set of peak candidates. Because large variations in the pulse morphology of the ICP signals exist the actual position of each of the three peaks is extremely variable. The complexity of data may lead to wrong or missed assignments in some instances.

In the alternative embodiment at Block 70, the position (p1, p2, p3) of the peaks is considered as a function f of the pulse signal. To this end, a regression model is exploited instead of the Gaussian priors during the peak designation to improve the accuracy of the process. One strength of using this model is that it exploits the values of the pulse itself during the peak assignment at Block 80. Another advantage is the ability of the framework to exploit powerful machine learning algorithms.

Finally, the designated peaks at Block 80 can be analyzed and morphological features can be extracted at Block 90 of FIG. 1. The various features can be used by treating physicians to evaluate the condition of the patient and make timely treatments to avoid potential future events.

Referring also to FIG. 2A and FIG. 2B, different metrics of the final designated pulse peaks can be identified and tracked over time. Such metrics can be clinically correlated with observed conditions in previous patients with different types of injuries or neurological conditions. In some settings, tracking ICP pulse morphological changes in a near real-time fashion can lead to forecasting intracranial pathological changes. For example, ICP pulses originate from blood pressure along the cerebral vasculature. A particular configuration of sub-peaks in an ICP pulse is influenced by arterial, capillary, and venous blood pressure pulses, as well as their interactions with the three major intracranial compartments, including the cerebral vasculature, the brain tissue, and the cerebrospinal fluid circulatory system. Therefore, the ICP pulse morphological changes may provide good indications of changes in any of these three compartments. These morphological changes can be caused by a variety of pathological events such as the narrowing cerebral arteries (vasospasm) after a subarachnoid hemorrhage and the development of mass-occupying lesions after a brain injury.

In the embodiment shown in FIG. 2, twenty four metrics have been identified that can be monitored and evaluated to make clinical decisions in a specific case or evaluated to identify patterns when compared with data from previous patients with characteristic conditions as illustrated in the Examples below. Libraries of patient peak and metric profiles can be assembled and stored to be available for future reference. Identified patterns and correlations of metrics with observed physiological symptoms and conditions can allow a treating physician to identify physical conditions, foretell events in evolving conditions and to provide prophylactic treatment. It can be seen that the clinical value of the morphological properties extracted by the invention provide more information than the mean ICP, which is currently used in clinical practice.

Embodiments of the present invention are described with reference to flowchart illustrations of methods and systems according to embodiments of the invention. These methods and systems can also be implemented as computer program products. In this regard, each block or step of a flowchart, and combinations of blocks (and/or steps) in a flowchart, can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts support combinations of means for performing the specified functions, combinations of steps for performing the specified functions, and computer program instructions, such as embodied in computer-readable program code logic means, for performing the specified functions. It will also be understood that each block of the flowchart illustrations, and combinations of blocks in the flowchart illustrations, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s).

The invention may be better understood with reference to the accompanying examples, which are intended for purposes of illustration only and should not be construed as in any sense limiting the scope of the present invention as defined in the claims appended hereto.

### Example 1

In order to demonstrate the functionality of the invention and the general principles behind the refining ICP Peak sub-transformations, comparative reconstructions of a phantom were conducted. In the embodiments tested, N signal segments were selected from the recoded ICP and ECG signals from 66 patients, including 32 females and 34 males, who were seen as inpatients at the University of California, Los Angeles (UCLA) Adult Hydrocephalus Center for various ICP-related conditions. The average age of these patients is 61 with their ages ranging from 14 to 94 years old.

During their hospitalization, the patients received continuous ICP monitoring for the clinical purpose using Codman intraparenchymal microsensors (Codman and Schurtleff, Raynaud, Mass.) situated in the right frontal lobe. Simultaneous cardiovascular monitoring was also performed using the bedside GE monitors. ICP and lead II of ECG signals were archived using either a mobile cart at the bedside that was equipped with the PowerLab TM SP-16 data acquisition system (ADInstruments, Colorado Springs, Colo.) or the BedMaster system that collects data from the GE Unity Network to which the bedside monitors were connected. Signal files in this archive were transformed into the Chart Binary file format for further processing. A total of N=153 signal segments, each of which is approximately 5-h long, were randomly selected, at an interval of 12 h, without avoiding noisy regions from these datasets. These 153 signal segments were subsequently processed according to the protocol set forth in FIG. 1.

A sequence length of 3 min was used in the MOCAIP algorithm, i.e., a dominant ICP pulse was generated for every 3-min recording. This choice resulted in a total of 14,230 raw dominant pulses. The number of non-artifactual dominant pulses was 13,371 accounting for 93.96% of identified dominant pulses.

Nonartifactual pulse recognition was demonstrated to be successful with an overall accuracy of 97.84%. This is partly due to the adoption of a reference library of non-artifactual ICP pulses and partly due to the use of the pulse clustering. Designation of P₁, P_{2 and} P₃ achieved an overall accuracy around 85% for P₃ and an accuracy approaching 90% for P₁. This may reflect the fact that P₂ and P₃ have a larger variability and that more candidate peaks are detected at later portions of an ICP pulse.

The MOCAIP algorithm was demonstrated to be able to accurately identify, from continuous ICP recordings, non-artifactual dominant ICP pulses for analysis and to satisfactorily detect and designate individual peaks in an ICP pulse.

### Example 2

An alternative embodiment of the system and method for detecting and designating individual subpeaks of ICP pulses from continuous ICP data was conducted using a singular value decomposition (SVD) technique as an alternative to the correlation based approach utilized in recognizing valid ICP pulses shown in FIG. 1. A comparative analysis of the valid ICP recognition using the SVD technique and the correlation based method demonstrated a significant improvement in terms of accuracy (61.96% reduction in false positive rate while keeping true positive rate as high as 99.08%) and computational time (91.14% less time consumption).

Using the same raw data set as used in Example 1, the extracted ICP and ECG signal segments were subsequently processed by MOCAIP and a dominant ICP pulse was generated for every 3 minutes of recording resulting in 14903 raw dominant pulses. All these 14903 dominant pulses were assessed by visual inspection and manually annotated as a valid ICP pulse (a typical triphasic ICP pulse or a non-valid ICP pulse caused by noise or artifacts or wrong QRS detection). As a result of this assessment, 13611 were annotated as valid pulses accounting for 91.33% of total dominant pulses.

The construction of the original reference library of valid ICP pulses which were used in the MOCAIP was conducted. Up to 10 validated dominant ICP pulses were selected from each of the 158 signal segments in a completely random fashion. This resulted in 1440 valid ICP pulses with the mean ICP of 3.1 plus or minus 7.2 mmHg. The mean amplitude of these ICP pulse was 6.6 plus or minus 3.3 mmHg.

To perform SVD on the original ICP reference library, we chose M as the 90th percentile of the lengths of pulses in the library. After resizing and normalizing all the pulses, a singular value decomposition on the matrix A 428×1440 was performed. The effective reference library was determined to be 18 pulses. The technique for recognizing valid ICP pulses using the singular value decomposition improved the correlation-based approach used in MOCAIP algorithm in terms of both accuracy and computational cost. In addition, this method has low sensitivity to the choice of number of bases in the reduced-noise signal space, the selection and number of ICP pulses to perform initial SVD. Finally, the proposed method may be potentially applicable to validate pulsatile physiological signals other than ICP pulses, e.g. ABP pulses and pulse Oximetry signals.

### Example 3

To illustrate the use of intracranial pressure (ICP) pulse morphological metrics to classify cerebral blood flow (CBF) into low and normal groups, forty-four acutely brain injured patients with ICP monitoring and daily ¹³³Xenon CBF were studied. Patient ICP recordings were time-aligned with the CBF measurements so that a one-hour ICP segment near the CBF measurement was obtained. Each of these recordings was processed by the Morphological Cluster and Analysis of Intracranial Pressure (MOCAIP) algorithm to extract pulse morphological metrics.

Although the full set of MOCAIP metrics illustrated in FIG. 2 can be used as input features to a classifier to separate different cerebral perfusion states, correlations exist among different MOCAIP metrics leading to redundancies if they are all used as input features. In addition, it is beneficial to use a minimal set of MOCAIP metrics to avoid unnecessarily complicating the classification. The challenge is that no prior knowledge exists with regard to what the relevant MOCAIP metrics are for characterizing cerebral perfusion states or symptoms.

Due to the lack of accurate prior knowledge regarding the pathophysiological implication of each of the MOCAIP metrics and their interactions, advanced data exploration tools that include global optimization, regularized quadratic classifier, and bootstrapping cross-validation techniques were used to design an experiment that requires minimal subjective choices of parameter values, e.g., which morphological metrics to use. Accordingly a two-class classification experiment where a threshold value based on cerebral blood flow (CBF) to designate the perfusion state was designed. After running the MOCAIP algorithm, a regularized quadratic classifier was trained using an optimization process, which resulted in the determination of the optimal combination of the MOCAIP metrics and the CSF drainage rate as well as the optimal parameters controlling the degree of regularization (w₁ and w₂). Under this optimal setup, a further cross-validation using a bootstrapping approach was conducted to obtain various performance metrics of the classification. In addition to the optimal subset of MOCAIP metrics, the performance of using the full set of MOCAIP metrics as feature vector using the same bootstrapping procedure were tested as well. In this case, optimal values of w₁ and w₂ were found using a differential evolution algorithm as well.

Most metrics that are shown in FIG. 2 were not selected. Only Lₜ, L₁, L₂, Lₓ, dP₁₃, and Curv₃ were selected for the majority of six independent runs (n 5). In addition to these six MOCAIP metrics, there are a few metrics, including dP₁₂, dP₃, diasP, mICP, and Curv₂₃, that were selected at least once but less than four times.

One of the findings from the classification experiment is that the elevation of the third peak of an ICP pulse may indicate low global cerebral perfusion. This is both visually confirmed from pulse graphs and by the fact that dP₁₃ was selected in all six independent runs of the experiment, which is significantly larger for the low CBF group.

While there appears to be an association between P₃ elevation and low global cerebral perfusion, further physiological studies are needed fully explain this observation. The origin of the P₃ has been largely attributed to the cerebral venous circulation. Therefore, an elevation of P₃ may indicate some pathological changes in the cerebral venous bed or elevated cerebral venous pressure, which consequently leads to the reduction of the cerebral perfusion pressure and causes a global cerebral perfusion deficit.

The metrics, including Lₜ, L₁, L₂, and Lₓ, were also included in the sub-group of classifier features in addition to the metrics that reflect P₃ elevation. The inclusion of Lₜ in the classification process can be probably explained by the fact that it measures the timing difference between ECG QRS peak and the onset of ICP pulse, which is significantly influenced by systemic arterial blood pressure. Therefore, Lₜ is a relevant measure as it contains information about the driving pressure of the cerebral blood flow.

One important implication of this study is that the system may be used to enable a prospective study where one uses ICP pulse morphological changes, which can be conveniently tracked, to actively trigger more detailed cerebral vascular, neuro-electrical, brain imaging and metabolism studies so that more accurate explanations can be found. Furthermore, attention to ICP pulse morphology in addition to the mean ICP may offer a practical monitoring practice to physicians for probing the functional integrality of the cerebral vasculature including the cerebral venous bed.

Therefore, the ICP pulse morphology analysis method of the present invention can be used to show that low global cerebral blood perfusion may be detected by using a set of ICP pulse morphological metrics through a trained pattern recognizer.

### Example 4

The system and methods of the present invention were used to derive 24 metrics characterizing morphology of ICP pulses and tested the hypothesis that pre-intracranial hypertension (pre-IH) segments of ICP can be differentiated, using these morphological metrics, from control segments that were not associated with any ICP elevation.

Thirty six 36 subjects were selected from 38 patients undergoing continuous intracranial pressure monitoring for: 1) headache evaluation in patients with suspected idiopathic intracranial hypertension or shunt malfunction, and 2) management of adult slit ventricle syndrome in which CSF flow from an externalized CSF shunt was purposefully stopped and 3) pre- or post-treatment of Chiari. Spontaneous intracranial hypertension can occur for all three patient populations. The ICP recordings were screened to identify episodes of intracranial hypertension defined as elevated ICP (>20 mmHg) over a period of at least five minutes. A total of 70 Pre-IH(0), 67 Pre-IH(5), 66 Pre-IH(10), 62 Pre-IH(15), and 54 Pre-IH(20) segments were generated.

In addition, a global optimization algorithm was used to effectively find the optimal sub-set of these morphological metrics to achieve better classification performance as compared to using full set of MOCAIP metrics.

The results showed that Pre-IH segments, using the optimal sub-set of metrics found by the differential evolution (DE) algorithm, can be differentiated from control segments at a specificity of 97% and sensitivity of 78% for those Pre-IH segments 5 minutes prior to the ICP elevation. While the sensitivity decreased to 68% for Pre-IH segments 20 minutes prior to ICP elevation, the high specificity remained. The performance using the full set of MOCAIP metrics was shown inferior to results achieved using the optimal sub-set of metrics. This demonstrated that advanced ICP pulse analysis combined with machine learning could potentially lead to the forecasting of ICP elevation so that a proactive ICP management could be realized based on accurate forecasts.

From the foregoing it can be seen that the present invention can be embodied in various ways, including, but not limited to, the following:

1. A method for extracting morphological features from intracranial pressure pulses, comprising: acquiring intracranial pressure pulse data of a patient from at least one sensor; refining the acquired pulse data with a computer and programming to produce refined pulse data; and determining peaks and metrics from said refined pulse data.

2. A method as recited in embodiment 1, wherein said acquired intracranial pressure pulse data comprises simultaneously recorded intracranial pressure (ICP) pulse and electrocardiogram (ECG) sensor data.

3. A method as recited in embodiment 1, wherein said refining of said acquired intracranial pressure pulse data comprises: segmenting continuously acquired intracranial pressure pulse data into a sequence of individual intracranial pressure pulses; clustering said sequences of segmented pulses to produce a plurality of refined pulses.

4. A method as recited in embodiment 3, further comprising: validating said refined pulses; and eliminating refined pulses that are not accurate intracranial pressure pulses.

5. A method as recited in embodiment 4, wherein said refined pulses are validated by a singular value decomposition algorithm.

6. A method as recited in embodiment 4, wherein said validation of said refined pulses comprises correlating said refined pulses with a library of previously validated ICP pulses.

7. A method as recited in embodiment 1, further comprising: selecting a final refined pulse from said refined pulses for analysis using an nonlinear regression model.

8. A method as recited in embodiment 1, further comprising: comparing said determined peaks and metrics from said refined intracranial pressure pulse data of a patient with a library of peak and metric profiles of prior patients.

9. A method as recited in embodiment 1, further comprising: recording pulse peak and metric data over time for a plurality of patients; correlating said pulse peak and metric data with physical and symptom data of each patient to produce a profile; forming a reference library of patient profiles; and comparing pulse peak and metric data of a current patient with patient profiles in said library of patient profiles.

10. A method for extracting morphological features from intracranial pressure pulses, comprising: obtaining intracranial pressure pulse data of a patient from a sensor; and processing said pressure pulse data with a computer, comprising: clustering said pulse data to produce a plurality of dominant pulses; validating said dominant pulses to eliminate false dominant pulses; detecting at least one subcomponent peak within said dominant pulses; designating final peaks and metrics of said dominant pulses; and analyzing said designated peaks and metrics.

11. A method as recited in embodiment 10, further comprising segmenting continuously obtained intracranial pressure pulse data into a sequence of individual intracranial pressure pulses.

12. A method as recited in embodiment 10, wherein said obtained intracranial pressure pulse data comprises simultaneously recorded intracranial pressure (ICP) pulse and electrocardiogram (ECG) sensor data.

13. A method as recited in embodiment 10, wherein said validation of said dominant pulses comprises comparing said dominant pulses with a library of previously validated ICP pulses.

14. A method as recited in embodiment 10, further comprising: clustering said dominant pulses to provide a set of clustered dominant pulses to be used for peak detection.

15. A method as recited in embodiment 10, wherein said designation of said final peaks comprises using a Gaussian prior of the distribution of each peak to designate at least one final peak.

16. A method as recited in embodiment 1, wherein said designation of said final peaks comprises using a nonlinear regression model.

17. A method as recited in embodiment 10, further comprising: monitoring said peaks and metrics obtained from said intracranial pulse data of a patient over a course of time; and comparing said peaks and metrics data with library of peaks and metrics to identify patterns of peaks and metrics.

18. A method for extracting morphological features from intracranial pressure pulses for patient treatment, comprising: acquiring intracranial pressure pulse data from a patient from a plurality of intracranial pressure (ICP) pulse and electrocardiogram (ECG) sensors; processing said intracranial pressure pulse data with a computer, comprising: clustering said pulse data to produce a plurality of dominant pulses; validating said dominant pulses to eliminate false dominant pulses; detecting at least one subcomponent peak within said dominant pulses; designating final peaks and metrics of said dominant pulses; and analyzing said designated peaks and metrics; comparing said analyzed and designated peaks and metrics of the patient with analyzed and designated intracranial pressure pulse peaks and metrics of one or more previous patients; and predicting possible physiological conditions and events of the patient from said comparison of said peaks and metrics.

19. A method as recited in embodiment 18, further comprising: recording final intracranial pressure pulse peaks and metrics obtained from intracranial pulse data of a patient over a course of time; correlating patient symptoms and conditions with said pulse peaks and metrics over said course of time; and forming a profile of correlated data for comparison with current patient data.

20. A method as recited in embodiment 19, further comprising: compiling a library of patient profiles; and identifying patterns of correlated symptoms, pulse peaks and metrics and time.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art. In any appended claims, reference to an element in the singular is not intended to mean “one and only one” unless explicitly so stated, but rather “one or more.” All structural, chemical, and functional equivalents to the elements of the above-described preferred embodiments that are known to those of ordinary skill in the art are expressly incorporated herein by reference and are intended to be encompassed by the present disclosure. Moreover, it is not necessary for a device or method to address each and every problem sought to be solved by the present invention, for it to be encompassed by the present disclosure. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for extracting morphological features from intracranial pressure pulses, comprising:
acquiring intracranial pressure pulse data of a patient from at least one sensor;
refining the acquired pulse data with a computer and programming to produce refined pulse data; and
determining peaks and metrics from said refined pulse data.

2. A method as recited in claim 1, wherein said acquired intracranial pressure pulse data comprises simultaneously recorded intracranial pressure (ICP) pulse and electrocardiogram (ECG) sensor data.

3. A method as recited in claim 1, wherein said refining of said acquired intracranial pressure pulse data comprises:
segmenting continuously acquired intracranial pressure pulse data into a sequence of individual intracranial pressure pulses;
clustering said sequences of segmented pulses to produce a plurality of refined pulses.

4. A method as recited in claim 3, further comprising:
validating said refined pulses; and
eliminating refined pulses that are not accurate intracranial pressure pulses.

5. A method as recited in claim 4, wherein said refined pulses are validated by a singular value decomposition algorithm.

6. A method as recited in claim 4, wherein said validation of said refined pulses comprises correlating said refined pulses with a library of previously validated ICP pulses.

7. A method as recited in claim 1, further comprising:
selecting a final refined pulse from said refined pulses for analysis using an nonlinear regression model.

8. A method as recited in claim 1, further comprising:
comparing said determined peaks and metrics from said refined intracranial pressure pulse data of a patient with a library of peak and metric profiles of prior patients.

9. A method as recited in claim 1, further comprising:
recording pulse peak and metric data over time for a plurality of patients;
correlating said pulse peak and metric data with physical and symptom data of each patient to produce a profile;
forming a reference library of patient profiles; and
comparing pulse peak and metric data of a current patient with patient profiles in said library of patient profiles.

10. A method for extracting morphological features from intracranial pressure pulses, comprising:
obtaining intracranial pressure pulse data of a patient from a sensor; and
processing said pressure pulse data with a computer, comprising:
clustering said pulse data to produce a plurality of dominant pulses;
validating said dominant pulses to eliminate false dominant pulses;
detecting at least one subcomponent peak within said dominant pulses;
designating final peaks and metrics of said dominant pulses; and
analyzing said designated peaks and metrics.

11. A method as recited in claim 10, further comprising segmenting continuously obtained intracranial pressure pulse data into a sequence of individual intracranial pressure pulses.

12. A method as recited in claim 10, wherein said obtained intracranial pressure pulse data comprises simultaneously recorded intracranial pressure (ICP) pulse and electrocardiogram (ECG) sensor data.

13. A method as recited in claim 10, wherein said validation of said dominant pulses comprises comparing said dominant pulses with a library of previously validated ICP pulses.

14. A method as recited in claim 10, further comprising:
clustering said dominant pulses to provide a set of clustered dominant pulses to be used for peak detection.

15. A method as recited in claim 10, wherein said designation of said final peaks comprises using a Gaussian prior of the distribution of each peak to designate at least one final peak.

16. A method as recited in claim 1, wherein said designation of said final peaks comprises using a nonlinear regression model.

17. A method as recited in claim 10, further comprising:
monitoring said peaks and metrics obtained from said intracranial pulse data of a patient over a course of time; and
comparing said peaks and metrics data with library of peaks and metrics to identify patterns of peaks and metrics.

18. A method for extracting morphological features from intracranial pressure pulses for patient treatment, comprising:
acquiring intracranial pressure pulse data from a patient from a plurality of intracranial pressure (ICP) pulse and electrocardiogram (ECG) sensors;
processing said intracranial pressure pulse data with a computer, comprising:
clustering said pulse data to produce a plurality of dominant pulses;
validating said dominant pulses to eliminate false dominant pulses;
detecting at least one subcomponent peak within said dominant pulses;
designating final peaks and metrics of said dominant pulses; and
analyzing said designated peaks and metrics;
comparing said analyzed and designated peaks and metrics of the patient with analyzed and designated intracranial pressure pulse peaks and metrics of one or more previous patients; and
predicting possible physiological conditions and events of the patient from said comparison of said peaks and metrics.

19. A method as recited in claim 18, further comprising:
recording final intracranial pressure pulse peaks and metrics obtained from intracranial pulse data of a patient over a course of time;
correlating patient symptoms and conditions with said pulse peaks and metrics over said course of time; and
forming a profile of correlated data for comparison with current patient data.

20. A method as recited in claim 19, further comprising:
compiling a library of patient profiles; and
identifying patterns of correlated symptoms, pulse peaks and metrics and time.
